(19) United States
(12) Patent Application Publication
(10) Pub. No.: US 2005/0084550 A1
(43) Pub. Date: Apr. 21, 2005

(54) Weight reduction and maintenance method for animals

(75) Inventor: James H. Brown, Scottsdale, AZ (US)

Correspondence Address: HOFFMANN & BARON, LLP, 6900 JERICHO TURNPIKE, SYOSSET, NY, 11791, US

(73) Assignee: International Flora Technologies

(21) Appl. No.: 11/005,430

(22) Filed: Dec. 6, 2004

Related U.S. Application Data
(63) Continuation of application No. 09/948,215, filed on Sep. 7, 2001, now abandoned.

(51) Int. Cl. A61K 35/78; A61K 31/225; A61K 31/7024

(52) U.S. Cl. 424/757; 514/42; 514/547

(57) ABSTRACT

Disclosed is a “Pet Food Formulation System” (PFFS) that is a “treatment system” that results in loss of weight by pets consuming ad libitum, or in controlled rations, a reduced calorie pet food. The PFFS will comprise the use of jojoba meal (JM) in the form of whole fat jojoba seed flour (WFJSF), expeller pressed jojoba seed meal, and/or solvent extracted jojoba seed meal, in combination with simmondsin concentrate (SC) and jojoba oil (JO), such combinations designed to both reduce the quantity of pet food consumed by the pet and at the same time reduce the level of calories contributed by the pet food that is consumed. Simmondsin concentrate and the natural simmondsin content of jojoba meal contained in a pet food for consumption by either dogs or cats serves to reduce pet appetite and therefore the amount of food that the pet is inclined to consume. The reduced food consumption is not due to reduced acceptability of the food by the pet, rather through the mechanism of reduced appetite produced by the simmondsin in the pet food.

### FIELD OF THE INVENTION

The present invention relates to a composition that is useful for weight reduction and/or maintenance in animals, preferably companion animals such as dogs, cats and the like. More specifically, the present invention relates to pet food compositions that include both an appetite suppressant and a calorie replacement, which are obtained from natural botanical sources. Methods of treating overweight animals, and weight maintenance using the disclosed compositions are also disclosed.

### BACKGROUND

In an affluent society obesity is the most common nutritional disease of animals such as humans, but especially of companion pets such as dogs and cats. Obesity generally is considered present when body weight of the animals, such as a companion pet, is 15% or more than the optimum weight value, which is the point at which health problems are increasingly manifest with increasing weight. Obesity exceeds by far all deficiency diseases combined. For example, it has been reported that, in affluent societies, from 24% to 44% of the companion dogs are obese. Generally speaking, the incidence of obesity in companion pets increases with the age of the pet. Similar to humans, as the animals age, exercise decreases, body fat increases and the amount of lean body mass decreases.

Because obesity develops gradually, a companion pet owner may be unaware of the overweight condition until it is called to his or her attention. Rarely is an animal presented to a veterinarian solely for the problem of obesity, but instead because of shortness of breath, routine immunizations, or arthritic or rheumatic symptoms. It is not uncommon that obesity is the predisposing cause of the condition noticed by the owner, although the obesity itself may not be noticed. For example, in one study it was observed that nearly one-third of the owners of obese dogs did not realize that their dogs were overweight.

The cause of obesity in companion pets is quite simple—energy or calorie intake in excess of that utilized. However, the factors causing this are not quite so simple. Some animals are known to be “easy keepers”. That is, they become overweight while being fed commercial pet foods in amounts adequate for normal adult maintenance, and in the same amount and manner as their companions who may maintain optimum weight.

In most instances, in companion pet obesity there are two stages—an initial phase and a static phase. The basic cause of the initial phase is a dietary energy intake in excess of that utilized, resulting in a positive energy balance which is deposited as fat. In the static phase, dietary intake is reduced in accordance with energy needs so that body weight remains constant in the obese state. Thus, the amount of food required for maintaining the animal in the obese state is no greater, and in fact is often less, than that required for maintaining the normal, non-obese state.

Thus, the only successful way for reducing companion animals is a drastic food or calorie intake reduction, or a combination of both. However, none of the diets currently available have proven satisfactory to many people or pets for reasons including increased stool volumes, reduced palatability, poor hair coats as a result of the diets' low fat content, but primarily because the diets do not sufficiently decrease the animals' hunger. When this occurs, the companion pets often scavenge and/or beg for food because of the sensation of constant hunger. As a result, the animal finds additional food, or pet owners feed the animal to stop the begging with the result being that no weight reduction occurs.

In short, it can be seen that for companion pets successful weight reduction involves not only decreased food intake, but as well an interdisciplinary, psychological treatment that involves the interrelationship between the companion animal and its human owner. This complex psychological interdependency makes weight reduction in companion pets even more difficult than it otherwise might be. In short, success at pet weight reduction involves initially convincing the owner that weight reduction is needed for the long-term health of their pet; secondly, the animal's food and calorie intake must be decreased for a sustained and regular period of time sufficient for weight reduction to occur; and third, the animal must be inhibited from constant scavenging and/or begging which tempts the owner to give in and increase the food intake to stop the begging.

In the past, certain drugs have been used in the treatment of obesity in mammals, including companion pets. These include drugs which decrease appetite such as amphetamines, drugs which cause nausea, decrease intestinal absorption, or increase metabolic rate such as thyroid hormones, and finally, drugs which either tranquilize or act as diuretics. None of the above drugs are generally effective. They often cause side effects, and tests of most have shown that such drugs are not only expensive, but also ineffective in that when ad libitum fed with food, the animals often tend to avoid the food that contains the drug.

It therefore can be seen that there is a real and continuing need for a treatment for overweight mammals, and especially companion pets, which is safe, efficacious, and which can successfully result in obesity reduction without changing the animal's behavioral patterns to such an extent that its relationship with its owner is changed. There is a further need for a treatment that safely and sufficiently inhibits hunger or induces satiety in the animals.

This invention has, as its primary objective, the fulfillment of these needs.

### SUMMARY OF INVENTION

It is an object of the present invention to provide a weight loss or maintenance method and food formulation comprising an effective amount of an appetite suppressant; and an effective amount of a reduced calorie contribution lipid substitute.

It is another object of the present invention to provide a method and food formulation as above wherein the appetite suppressant further comprises a short-term appetite suppressant and a long-term appetite suppressant.

It is yet another object of the present invention to provide a method and food formulation as above wherein the short-term appetite suppressant is comprised of a simmondsin compound.

It is a further object of the present invention to provide a method and food formulation as above wherein the simmondsin compound of the short-term appetite suppressant is a concentrated simmondsin compound that is more concentrated than the naturally occurring concentration found in jojoba seeds or jojoba meal.

It is yet a further object of the present invention to provide a method and food formulation as above wherein the long-term appetite suppressant is comprised of a simmondsin compound.

It is still yet a further object of the present invention to provide a method and food formulation as above wherein the simmondsin compound of the long-term appetite suppressant is in the form of jojoba meal.

It is another object of the present invention to provide a method and food formulation as above wherein the reduced calorie contribution lipid substitute is isolated from a natural botanical product.

It is yet another object of the present invention to provide a method and food formulation as above wherein the natural product is jojoba seed.

It is still yet another object of the present invention to provide a method and food formulation as above wherein the reduced calorie contribution lipid substitute is provided in at least a liquid form.

It is a further object of the present invention to provide a method and food formulation as above wherein the liquid form of the reduced calorie contribution lipid substitute is jojoba oil.

It is still a further object of the present invention to provide a method and food formulation as above wherein the reduced calorie contribution lipid substitute is provided in at least a solid form.

It is still yet a further object of the present invention to provide a method and food formulation as above wherein the solid form of the reduced calorie contribution lipid substitute is whole or partially fatted jojoba seed flour.

It is another object of the present invention to provide a method and food formulation as above wherein the reduced calorie contribution lipid substitute is provided in at least a liquid form and at least a solid form.

It is yet another object of the present invention to provide a method and food formulation as above wherein the appetite suppressant is further comprised of a component that is a concentrated form of simmondsin compound and a component that is a naturally occurring concentration of simmondsin compound and the reduced calorie contribution lipid substitute is provided in at least a liquid form and at least a solid form.

It is still yet another object of the present invention to provide a method and food formulation as above wherein the liquid form of the reduced calorie contribution lipid substitute is jojoba oil and the solid form of the reduced calorie contribution lipid substitute is whole or partially fatted jojoba seed flour.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §1 12, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DETAILED DESCRIPTION OF PREFERRED EMBODIMENTS

The present invention is useful for weight reduction and/or maintenance in animals, such as companion pets. The present invention is a food composition, the “Pet Food Formulation System” (PFFS), that is a “treatment system” that results in loss of weight by animals, such as pets, consuming ad libitum or a controlled rationing of a reduced calorie pet food. A preferred PFFS comprises a botanical based appetite suppressant and a botanical based reduced calorie contribution lipid substitute. A most preferred PFFS comprises jojoba meal in the form of whole fat jojoba seed flour (WFJSF), expeller pressed jojoba seed meal, and /or solvent extracted jojoba seed meal (generally referred to as partially fatted jojoba seed flour or PFJSF), in combination with simmondsin concentrate (SC), and jojoba oil (JO), which are designed to both reduce the quantity of pet food consumed by the pet and, at the same time, reduce the level of calories contributed by lipids in the pet food that is consumed.

Jojoba, Simmondsia chinensis, is native to the desert Southwestern United States and Mexico. It is currently being grown on about 8,500 hectares worldwide. Jojoba seeds contain a unique wax ester in a lipid phase (oil) that is 40 to 60% of the seed weight. This oil is currently used in cosmetics and lubricants. The remainder of the seed, defatted seed meal, which still contains a fraction of jojoba oil, is not commercially used as much as the extracted oil, even though it contains about 25% crude protein after the oil is removed. In addition to the crude protein, the defatted seed meal contains sugars and 11-15% of a unique group of natural products, all structurally related to simmondsin and its analogues. Cokeleare et al. (1995, Ind. Crops Prod., 4:91-96) have shown that simmondsin and its analogues are an effective appetite suppressant that reduces food intake in mice, rats and chickens. Thus, jojoba seed meal has been used for its simmondsin content to regulate the food intake of animals, see for example U.S. Pat. Nos. 5,962,043 and 6,245,364, which are incorporated herein by reference. Therefore the reduced food intake by animals consuming jojoba meal in their food is not due to reduced acceptability of the food by the animal, rather through the mechanism of appetite suppression produced by the simmondsin in the food.

Simmondsin delivered in the form of simmondsin concentrate serves to induce an almost immediate decrease in the appetite of the pet as the pet begins to consume the food. The simmondsin in the WFJSF or PSJFS serves as a time-release mechanism for simmondsin, wherein the release time depends upon the WFJSF or PSJFS granulation size and the duration depends upon the concentration of the WFJSF or PSJFS. Thus, a combination of both simmondsin concentrate and WFJSF and/or PSJFS in a pet food formulation can be designed to deliver both an immediate decrease in appetite as well as a longer term or delayed decrease in appetite.

In the most preferred embodiment, the reduced calorie contribution lipid substitute, preferably jojoba oil (or its derivatives such as hydrogenated jojoba oil, partially saturated jojoba oil, and the like) that is added neat, or as the natural jojoba oil content of WFJSF and/or PSJFS, is a reduced calorie contribution lipid substitute or partial lipid substitute for ordinary vegetable oil typically used in pet food formulations. Triglyceride vegetable oils typically comprise the major lipid component of such formulations. By substituting jojoba oil for a portion of the triglyceride vegetable oil in the present invention the net caloric contribution of the lipid component of the pet food formulation is reduced and therefore the total calories realized by any animal consuming such formulation is also reduced.

Jojoba oil (or Jojoba waxes) are liquid above 10° C. and are a mixture of linear esters of monounsaturated, long-chain fatty acids and long-chain primary fatty alcohols. While jojoba oils have a higher gross energy value (10.3 kcal/g) as compared to triglyceride vegetable oils or other triacylglycerol fats (9 kcal/g), they are poorly digested and result in a reduced calorie contribution (5.4 kcal/g) when included in a food formulation. Moreover, dietary jojoba oil has been found to lead to significant reduction in blood cholesterol. Finally, jojoba oil is resistant to oxidation, thereby prolonging the shelf live of the food formulation, as compared to most other triglyceride vegetable oil formulations.

Whole fatted jojoba seed flour (WFJSF) is the product of the grinding of whole jojoba seeds. The jojoba oil content of WFJSF is the same as that of jojoba seed; the natural jojoba oil content of jojoba seed may vary from as low as 40% to as high as 60% in some varieties of jojoba which would result in the WFJSF containing 40% to 60% jojoba oil. The particle size of the WFJSF can be controlled in the grinding operation so that fractions of 20/60 mesh and/or 60/100 mesh (or any other range of particle sizes that may be desired) are produced. The natural simmondsin content of the WFJSF is approximately 5% which is the same as the simmondsin content of unground jojoba seed. The natural simmondsin content in jojoba seed may vary from as low as 2% to as high as 8% in some varieties, which would result in flour containing 2% to 8% simmondsin.

In typical oilseed crushing facilities jojoba oil is extracted from jojoba seed by passing the seed through expeller presses or through equipment known as expanders. Both types of equipment result in a physical (rather than chemical) removal by pressing of jojoba oil from jojoba seed. Jojoba oil extracted from this physical pressing is one product of the operation, while expeller or expander presscake (EPC) is the other product of the operation. EPC is also known asjojoba “meal”. Simmondsin is not soluble in jojoba oil and therefore is concentrated in the EPC fraction of the output of the press. The particle size of the resulting EPC can be controlled in a grinding operation so that fractions of 20/60 mesh and/or 60/100 mesh (or any other range of particle sizes that may be desired) are produced. EPC typically contains 9% residual jojoba oil although the residual oil content can be varied through adjustment of the equipment so that the EPC contains as little as 5% to as much as 40% retained jojoba oil.

Following the expeller or expander pressing of jojoba oil from jojoba seed, EPC is typically passed through a solvent extraction operation in which a solvent such as hexane is used to extract any residual jojoba oil remaining in the EPC. Simmondsin is not soluble in hexane and is therefore concentrated in the solvent extracted press cake (SEPC) emerging from the solvent extraction operation. This SEPC is also known as solvent extracted jojoba meal. In efficient solvent extraction facilities the residual jojoba oil content of the SEPC will be near zero, but is usually about 1%. The particle size of the resulting SEPC can be controlled in a grinding operation so that fractions of 20/60 mesh and/or 60/100 mesh (or any other range of particle sizes that may be desired) are produced. For the purpose of this application, SEPC and EPC together will be referred to as partially fatted jojoba seed flour (PFJSF), the product of the grinding of expeller or expander pressed jojoba seed meal or solvent extracted jojoba seed meal.

Jojoba oil occurs naturally in jojoba seed in a range from about 40% to about 60%. Simmondsin occurs naturally in jojoba seed in a range of about 2% to about 8%. As jojoba oil is removed from jojoba seed by expeller or expander presses, solvent extraction equipment, or a combination of these different equipment, simmondsin is concentrated in the residual meal. By way of example, jojoba seed containing the minimum 40% jojoba oil and 2% simmondsin, after complete processing to remove jojoba oil would result in SEPC containing about 3% simmondsin. Likewise, jojoba seed containing the maximum 60% jojoba oil and 8% simmondsin, after complete processing to remove jojoba oil would result in SEPC containing about 20% simmondsin. Graph 1 illustrates the range of concentrations of jojoba oil and simmondsin naturally occurring in jojoba seed and the resulting concentration of simmondsin when all or some of the jojoba oil has been removed from the seed by the different methods.

Any one of the above examples, i.e. WFJSF, or PFJSF can be used in the PFFS as a vehicle for the delivery of jojoba oil or simmondsin, or for the delivery of both. For the purpose of this application WFJSF will be used in all examples although EPC, SEPC (PFJSF) or a combination of two or all three forms may be used depending upon the objectives of the formulator.

Optimum amounts of WFJSF, SC and JO in a pet food formulation depend upon the particular animal or pet and the condition targeted for treatment. As an example, a PFFS may be prepared that will result in pets consuming between 50% and 95% the amount of pet food ordinarily consumed. The caloric contribution of the PFFS consumed can be adjusted so that the pet realizes as little as 40% of the calories contained in a normal pet food with little decrease in lipid content. This range of pet food consumption and caloric contribution can be adjusted so that the pets experience rapid weight loss (3-7% or greater of body weight per week), controlled weight loss (3% or less of body weight loss per week), or for weight maintenance of the pet.

The following are examples of the use of combinations of WFJSF, SC and JO and ordinary vegetable oil in pet food formulations and are calculated so that the pet will realize various ratios of calories and simmondsin when the formulation is fed to the pet. The form of the simmondsin delivery vehicle, i.e. simmondsin concentrate or WFJSF, together with the granulation of the WFJSF, are calculated to provide a more immediate or long-term (or both) release of simmondsin to the pet, and therefore a more immediate or longer term (or both) decrease of appetite in the pet.

These examples do not take into account the caloric or nutrient content of other ingredients used in pet food formulations except that the assumption is made that the other ingredients in the formulation would contribute approximately 2% of the lipid fraction of the complete formulation. In these examples it is anticipated that a total lipid fraction of 12%, typical of a diet type pet food formulation, will result in a pet food with satisfactory taste for the pet although some dry pet foods may contain as much as 16% or more lipid component, typical for a regular pet food formulation.

Example Calculation Parameters:

WFJSF=50% JO and contains 5% simmondsin;

SC=50% simmondsin;

JO=contributes 5.4 Calories per gram;

Triglyceride vegetable oil=9 Calories per gram

WFJSF(1)=−20/60 mesh (coarse granulation)

WFJSF(2)=−60/100 mesh (fine granulation)

An ordinary formula containing 12% lipid fraction from triglyceride vegetable oil would deliver approximately 108 lipid calories per 100 gm of pet food; 16% lipid fraction would deliver approximately 144 lipid calories per 100 gm of pet food.

### EXAMPLE 1

Formula containing 1% simmondsin and 12% lipids. If the 12% lipids were from conventional lipids they would deliver 108 calories. With 10 grams or 83.3% of the lipid fraction coming from jojoba oil (either neat or from WFJSF) the diet results in a total of only 67.5 lipid calories contributed or 62.5% of a conventional diet. Further, 50% of the simmondsin content in this diet is provided from the simmondsin concentrate and 50% of the simmondsin content in this diet is provided from a combination of fine and coarse ground WFJSF; thereby providing approximately equal short-term and long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1), | 10.0 | 0.5 | 5.0 | 27.0 | 25.0 |
| (2) | | | | | |
| SC | 1.0 | 0.5 | 0.0 | | |
| JO | 5.0 | 0.0 | 5.0 | 22.5 | 20.8 |
| Vegetable | 0.0 | 0.0 | 0.0 | | |
| Oil | | | | | |
| Other | 84.0 | 0.0 | 2.0 | 18.0 | 16.7 |
| Ingredients | | | | | |
| Total | 100 | 1.0 | 12.0 | 67.0 | 62.5 |

### EXAMPLE 2

Formula containing 1% simmondsin and 12% lipids. If the 12% lipids were from conventional lipids they would deliver 108 calories. With 7 grams or 58.3% of the lipid fraction coming from jojoba oil (either neat or from WFJSF) the diet results in a total of only 82.2 lipid calories contributed or 76.7% of a conventional diet. Further, 90% of the simmondsin content in this diet is provided from the simmondsin concentrate and 10% of the simmondsin content in this diet is provided from fine ground WFJSF; thereby providing a stronger short-term appetite suppression compared to the long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(2) | 2.0 | 0.1 | 1.0 | 5.4 | 5.0 |
| SC | 1.8 | 0.9 | 0.0 | | |
| JO | 6.0 | 0.0 | 6.0 | 32.4 | 30.0 |
| Vegetable | 3.0 | 0.0 | 3.0 | 27.0 | 25.0 |
| Oil | | | | | |
| Other | 87.2 | 0.0 | 2.0 | 18.0 | 16.7 |
| Ingredients | | | | | |
| Total | 100 | 1.0 | 12.0 | 82.8 | 76.7 |

### EXAMPLE 3

Formula containing 1% simmondsin and 12% lipids. Further, 100% of the simmondsin content in this diet is provided from the simmondsin concentrate with none of the simmondsin content in this diet provided from WFJSF; thereby providing short-term appetite suppression with relatively little long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF | 0.0 | 0.0 | 0.0 | | |
| SC | 2.0 | 1.0 | 0.0 | | |
| JO | 0.0 | 0.0 | 0.0 | | |
| Vegetable | 10.0 | 0.0 | 10.0 | 90.0 | 83.3 |
| Oil | | | | | |
| Other | 88.0 | 0.0 | 2.0 | 18.0 | 16.7 |
| Ingredients | | | | | |
| Total | 100 | 1.0 | 12.0 | 108.0 | 100 |

### EXAMPLE 4

Formula containing 1% simmondsin and 12% lipids. If the 12% lipids were from conventional lipids they would deliver 108 calories. With 10 grams or 83.3% of the lipid fraction coming from jojoba oil (either neat or from WFJSF) the diet results in a total of only 72.0 lipid calories contributed or 67.7% of a conventional diet. Further, ⅓ of the simmondsin content in this diet is provided from the simmondsin concentrate and ⅔ of the simmondsin content in this diet is provided from coarse ground WFJSF; thereby providing a greater long-term appetite suppression relative to short-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1) | 2.0 | 0.1 | 1.0 | 5.4 | 5.0 |
| SC | 0.1 | 0.05 | 0.0 | | |
| JO | 9.0 | 0.0 | 9.0 | 48.6 | 45.0 |
| Vegetable | 0.0 | 0.0 | 0.0 | | |
| Oil | | | | | |
| Other | 88.9 | 0.0 | 2.0 | 18.0 | 16.7 |
| Ingredients | | | | | |
| Total | 100 | 0.15 | 12.0 | 72.0 | 67.7 |

### EXAMPLE 5

Formula containing 1% simmondsin and 12% lipids. If the 12% lipids were from conventional lipids they would deliver 108 calories. With 7 grams or 58.3% of the lipid fraction coming from jojoba oil (either neat or from WFJSF) the diet results a total of only 82.8 lipid calories or 76.7% of a conventional diet. Further, ⅓ of the simmondsin content in this diet is provided from the simmondsin concentrate and ⅔ of the simmondsin content in this diet is provided from coarse ground WFJSF; thereby providing a greater long-term appetite suppression relative to short-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1) | 2.0 | 0.1 | 1.0 | 5.4 | 5.0 |
| SC | 0.1 | 0.05 | 0.0 | | |
| JO | 6.0 | 0.0 | 6.0 | 32.4 | 30.0 |
| Vegetable | 3.0 | 0.0 | 3.0 | 27.0 | 25.0 |
| Oil | | | | | |
| Other | 88.9 | 0.0 | 2.0 | 18.0 | 16.7 |
| Ingredients | | | | | |
| Total | 100 | 0.15 | 12.0 | 82.8 | 76.7 |

### EXAMPLE 6

Formula containing 1% simmondsin and 12% lipids. If the 12% lipids were from conventional lipids they would deliver 108 calories. With none of the lipid fraction coming from jojoba oil (either neat or from WFJSF) the diet results in a total of 108.0 lipid calories contributed or 100% of a conventional diet. Further, 100% of the simmondsin content in this diet is provided from the simmondsin concentrate with none of the simmondsin content in this diet provided from WFJSF; thereby providing short-term appetite suppression with relatively little long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF | 0.0 | 0.0 | 0.0 | | |
| SC | 0.3 | 0.15 | 0.0 | | |
| JO | 0.0 | 0.0 | 0.0 | | |
| Vegetable | 10.0 | 0.0 | 10.0 | 90.0 | 83.3 |
| Oil | | | | | |
| Other | 89.7 | 0.0 | 2.0 | 18.0 | 16.7 |
| Ingredients | | | | | |
| Total | 100 | 0.15 | 12.0 | 108.0 | 100 |

### EXAMPLE 7

Formula containing 1% simmondsin and 12% lipids. If the 12% lipids were from conventional lipids they would deliver 108 calories. With 10 grams or 83.3% of the lipid fraction coming from jojoba oil (either neat or from WFJSF) the diet results in a total of only 72.0 lipid calories contributed or 66.7% of a conventional diet. Further, none of the simmondsin content in this diet is provided from the simmondsin concentrate and 100% of the simmondsin content in this diet is provided from a combination of coarse and fine ground WFJSF; thereby providing longer term appetite suppression with relatively little immediate appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1), | 8.0 | 0.4 | 4.0 | 21.6 | 20.0 |
| (2) | | | | | |
| SC | 0.0 | 0.0 | 0.0 | | |
| JO | 6.0 | 0.0 | 6.0 | 32.4 | 30.0 |
| Vegetable | 0.0 | 0.0 | 0.0 | | |
| Oil | | | | | |
| Other | 86.0 | 0.0 | 2.0 | 18.0 | 16.7 |
| Ingredients | | | | | |
| Total | 100 | 0.4 | 12.0 | 72.0 | 66.7 |

### EXAMPLE 8

Formula containing 1% simmondsin and 12% lipids. If the 12% lipids were from conventional lipids they would deliver 108 calories. With 4.0 grams or 33.3% of the lipid fraction coming from jojoba oil (either neat or from WFJSF) the diet results in a total of only 93.6 lipid calories contributed or 86.7% of a conventional diet. Further, 50% of the simmondsin content in this diet is provided from the simmondsin concentrate and 50% of the simmondsin content in this diet is provided from a combination of coarse and fine ground WFJSF; thereby providing approximately equal short-term and long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1), | 4.0 | 0.2 | 2.0 | 10.8 | 10.0 |
| (2) | | | | | |
| SC | 0.4 | 0.2 | 0.0 | | |
| JO | 2.0 | 0.0 | 2.0 | 10.8 | 10.0 |
| Vegetable | 6.0 | 0.0 | 6.0 | 54.0 | 50.0 |
| Oil | | | | | |
| Other | 87.6 | 0.0 | 2.0 | 18.0 | 16.7 |
| Ingredients | | | | | |
| Total | 100 | 0.4 | 12.0 | 93.6 | 86.7 |

### EXAMPLE 9

Formula containing 1% simmondsin and 12% lipids. If the 12% lipids were from conventional lipids they would deliver 108 calories. With 1 grams or 8.3% of the lipid fraction coming from jojoba oil (from WFJSF) the diet results in a total of only 104.4 lipid calories contributed or 96.7% of a conventional diet. Further, 75% of the simmondsin content in this diet is provided from the simmondsin concentrate and 25% of the simmondsin content in this diet is provided from fine ground WFJSF; thereby providing a stronger short-term appetite suppression compared to the long-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(2) | 2.0 | 0.1 | 1.0 | 5.4 | 5.0 |
| SC | 0.6 | 0.3 | 0.0 | | |
| JO | 0.0 | 0.0 | 0.0 | | |
| Vegetable | 9.0 | 0.0 | 9.0 | 81.0 | 75.0 |
| Oil | | | | | |
| Other | 88.4 | 0.0 | 2.0 | 18.0 | 16.7 |
| Ingredients | | | | | |
| Total | 100 | 0.4 | 12.0 | 104.4 | 96.7 |

### EXAMPLE 10

Formula containing 1% simmondsin and 12% lipids. If the 12% lipids were from conventional lipids they would deliver 108 calories. With 10 grams or 83.3% of the lipid fraction coming from jojoba oil (either neat or from WFJSF) the diet results in a total of only 72.0 lipid calories or 66.7% of a conventional diet. Further, none of the simmondsin content in this diet is provided from the simmondsin concentrate and 100% of the simmondsin content in this diet is provided from a combination of coarse and fine ground WFJSF; thereby providing long-term appetite suppression with relatively little short-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1), | 3.6 | 0.18 | 1.8 | 9.7 | 9.0 |
| (2) | | | | | |
| SC | 0.0 | 0.0 | 0.0 | | |
| JO | 8.2 | 0.0 | 8.2 | 44.3 | 41.0 |
| Vegetable | 0.0 | 0.0 | 0.0 | | |
| Oil | | | | | |
| Other | 88.2 | 0.0 | 2.0 | 18.0 | 16.7 |
| Ingredients | | | | | |
| Total | 100 | 0.18 | 12.0 | 72.0 | 66.7 |

### EXAMPLE 11

Formula containing 1% simmondsin and 12% lipids. If the 12% lipids were from conventional lipids they would deliver 108 calories. With 7 grams or 58.3% of the lipid fraction coming from jojoba oil (either neat or from WFJSF) the diet results a total of only 82.8 lipid calories contributed or 76.7% of a conventional diet. Further, 44% of the simmondsin content in this diet is provided from the simmondsin concentrate and 56% of the simmondsin content in this diet is provided from coarse ground WFJSF; thereby providing a slightly stronger long-term appetite suppression compared to the short-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1) | 2.0 | 0.1 | 1.0 | 5.4 | 5.0 |
| SC | 0.16 | 0.08 | 0.0 | | |
| JO | 6.0 | 0.0 | 6.0 | 32.4 | 30.0 |
| Vegetable | 3.0 | 0.0 | 3.0 | 27.0 | 25.0 |
| Oil | | | | | |
| Other | 88.84 | 0.0 | 2.0 | 18.0 | 16.7 |
| Ingredients | | | | | |
| Total | 100 | 0.18 | 12.0 | 82.8 | 76.7 |

### EXAMPLE 12

Formula containing 1% simmondsin and 12% lipids. If the 12% lipids were from conventional lipids they would deliver 108 calories. With 1 gram or 8.3% of the lipid fraction coming from jojoba oil (from WFJSF) the diet results in a total of only 104.4 lipid calories contributed or 96.7% of a conventional diet. Further, 44% of the simmondsin content in this diet is provided from the simmondsin concentrate and 56% of the simmondsin content in this diet is provided from coarse ground WFJSF; thereby providing a slightly stronger long-term appetite suppression compared to the short-term appetite suppression.

| | | | | | % of |
|---|---|---|---|---|---|
| | | | | | Conven- |
| | Quantity | Simmondsin | Lipid | Calories | tional |
| Ingredient | (%) | (%) | (%) | Contributed | Calories |
| WFJSF(1) | 2.0 | 0.1 | 1.0 | 5.4 | 5.0 |
| SC | 0.16 | 0.08 | 0.0 | | |
| JO | 0.0 | 0.0 | 0.0 | | |
| Vegetable | 9.0 | 0.0 | 9.0 | 81.0 | 75.0 |
| Oil | | | | | |
| Other | 88.84 | 0.0 | 2.0 | 18.0 | 16.7 |
| Ingredients | | | | | |
| Total | 100 | 0.18 | 12.0 | 104.4 | 96.7 |

Thus, it can be seen from the above representative examples, that the food formulations according to the present invention have three primary variables, a jojoba seed flour (either whole fatted jojoba seed flour, partially fatted jojoba seed flour, defatted jojoba seed flour or combinations if the three), simmondsin concentrate and jojoba oil. The remainder of the formulations comprise vegetable oil and other ingredients, as are commonly found in pet food formulations. The three primary variables, JM, SC and JO may range in concentration from 0-10%, 0-2%, and 0-9%, respectively, depending upon the weight loss/maintenance effect designed and depending upon the pet segment and or dietary condition being treated.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

1. A weight loss or maintenance food formulation comprising:
a. an effective amount of an appetite suppressant; and
b. an effective amount of a reduced calorie contribution lipid substitute.

2. The food formulation according to claim 1 wherein the appetite suppressant further comprises a short-term appetite suppressant and a long-term appetite suppressant.

3. The food formulation according to claim 2 wherein the short-term appetite suppressant is comprised of a simmondsin compound.

4. The food formulation according to claim 3 wherein the simmondsin compound of the short-term appetite suppressant is a concentrated simmondsin compound that is more concentrated than the naturally occurring concentration found in jojoba seeds or jojoba meal.

5. The food formulation according to claim 2 wherein the long-term appetite suppressant is comprised of a simmondsin compound.

6. The food formulation according to claim 5 wherein the simmondsin compound of the long-term appetite suppressant is in the form of jojoba meal.

7. The food formulation according to claim 1 wherein the reduced calorie contribution lipid substitute is isolated from a natural botanical product.

8. The food formulation according to claim 7 wherein the natural product is jojoba seed.

9. The food formulation according to claim 1 wherein the reduced calorie contribution lipid substitute is provided in at least a liquid form.

10. The food formulation according to claim 9 wherein the liquid form of the reduced calorie contribution lipid substitute is jojoba oil.

11. The food formulation according to claim 1 wherein the reduced calorie contribution lipid substitute is provided in at least a solid form.

12. The food formulation according to claim 11 wherein the solid form of the reduced calorie contribution lipid substitute is whole or partially fatted jojoba seed flour.

13. The food formulation according to claim 1 wherein the reduced calorie contribution lipid substitute is provided in at least a liquid form and at least a solid form.

14. The food formulation according to claim 13 wherein the liquid form of the reduced calorie contribution lipid substitute is jojoba oil.

15. The food formulation according to claim 13 wherein the solid form of the reduced calorie contribution lipid substitute is whole or partially fatted jojoba seed flour.

16. The food formulation according to claim 13 wherein the liquid form of the reduced calorie contribution lipid substitute is jojoba oil and the solid form of the reduced calorie contribution lipid substitute is whole or partially fatted jojoba seed flour.

17. The food formulation according to claim 1 wherein the appetite suppressant is further comprised of a component that is a concentrated form of simmondsin compound and a component that is a naturally occurring concentration of simmondsin compound and the reduced calorie contribution lipid substitute is provided in at least a liquid form and at least a solid form.

18. The food formulation according to claim 17 wherein the liquid form of the reduced calorie contribution lipid substitute is jojoba oil and the solid form of the reduced calorie contribution lipid substitute is whole or partially fatted jojoba seed flour.

19. A weight loss or maintenance food formulation comprising:
a jojoba oil ranging from 0 to 9% by weight of the food formulation;
b. a simmondsin compound that is more concentrated than the naturally occurring concentration found in jojoba seed and ranging from 0 to 2% by weight of the food formulation.
c. whole or partially fatted jojoba seed flour ranging from 0 to 10% by weight of the food formulation;
wherein the concentration of the jojoba oil and whole or partially fatted jojoba seed flour are not both 0 at the same time and the concentration of the concentrated simmondsin compound and the whole fatted jojoba seed flour are not both 0 at the same time.

20. A method for providing weight loss or maintenance to an animal comprising the step of providing to the animal a short-term appetite suppressant and a long-term appetite suppressant.

21. The method according to claim 20 wherein the short-term appetite suppressant is a simmondsin compound that is more concentrated that the naturally occurring concentration found in jojoba seed or jojoba meal.

22. The method according to claim 20 wherein the long-term appetite suppressant is a simmondsin compound that is in the naturally occurring concentration found in jojoba seed or jojoba meal.

23. The method according to claim 22 wherein the naturally occurring concentration of simmondsin compound is provided by at least whole or partially fatted jojoba seed flour.

24. The method according to claim 20 further including the step of providing to the animal a reduced calorie contribution lipid substitute.

25. The method according to claim 24 wherein the reduced calorie contribution lipid substitute is jojoba oil.

26. The method according to claim 25 wherein the jojoba oil is provided in at least a neat form.

27. The method according to claim 25 wherein the jojoba oil is provided by at least whole or partially fatted jojoba seed flour.

28. The method according to claim 25 wherein the jojoba oil is provided by at least neat jojoba oil and at least whole or partially fatted jojoba seed flour.

29. The method according to claim 24 wherein the short-term appetite suppressant is in a form more concentrated than the naturally occurring concentration found in jojoba seed and the long-term appetite suppressant is in a form that is in the naturally occurring concentration found in jojoba seed or jojoba meal; and the reduced calorie contribution lipid substitute is jojoba oil.

30. The method according to claim 29 wherein the jojoba oil is provided by at least neat jojoba oil and at least whole or partially fatted jojoba seed flour.
